(19) United States
(12) Patent Application Publication
(10) Pub. No.: US 2010/0203004 A1
(43) Pub. Date: Aug. 12, 2010

(54) COSMETIC COMPOSITION CONTAINING A TENSIONING AGENT AND AN ACRYLIC POLYMER

(75) Inventors: Jean-Thierry Simonnet, Cachan (FR); Guillaume Cassin, Villebon Sur Yvette (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'Oreal, Paris (FR)

(21) Appl. No.: 12/676,541

(22) PCT Filed: Oct. 28, 2008
(86) PCT No.: PCT/EP08/64612

§ 371 Date: Mar. 4, 2010

(51) Int. Cl. A61K 31/78; A61K 38/16; A61K 33/06; A61K 33/00; A61Q 19/08

(52) U.S. Cl. 424/78.03; 514/12; 424/684; 424/724

(57) ABSTRACT

The invention relates to a cosmetic composition comprising, in a physiologically acceptable medium, a skin-tensioning agent and an acrylic polymer containing a group of phosphorylcholine type. The invention also relates to a cosmetic skincare process, more particularly for facial skin, in particular wrinkled skin, comprising the application of the composition to the skin.

The present invention relates to a cosmetic composition, especially an anti-wrinkle composition, comprising, in a physiologically acceptable medium, a tensioning agent and an acrylic polymer containing a phosphorylcholine group.

The invention also relates to a cosmetic process for caring for wrinkled skin, for the purpose of attenuating wrinkles, comprising the application to the said skin of a composition as defined above.

During the ageing process, impairment of the structure and functions of the skin appears. The main clinical signs observed are the appearance of wrinkles and fine lines associated with slackening of the skin. A person skilled in the art knows that such slackening may be corrected immediately by applying a tensioning agent to the skin. This tensioning agent has the property of making the skin taut, and this tensioning effect makes it possible to smooth out the skin and to reduce the wrinkles and fine lines of the skin, or even make them disappear.

The use of numerous tensioning polymers for treating wrinkles is known to those skilled in the art at the present time. Acrylic-grafted silicone polymers or interpenetrating polymer networks for smoothing out wrinkles via a tensioning effect, described especially in documents EP 1 038 519 and FR 2 843 025, are recalled in particular.

Unfortunately, when these tensioning agents are formulated in the presence of hydrating polyols such as glycerol, their tensioning effect is considerably reduced, or may even disappear. Now, a need remains for a skincare composition that has efficient tensioning properties and moisturizing properties, which are especially suitable for treating skin with a tendency towards dryness.

Now, the Applicant has discovered that the introduction of an acrylic polymer containing a phosphorylcholine group into compositions comprising a tensioning agent makes it possible to conserve the tensioning properties of the composition, while at the same time imparting moisturizing properties.

One subject of the present invention is thus a cosmetic composition comprising, in a physiologically acceptable medium, a tensioning agent and an acrylic polymer containing a phosphorylcholine group.

A subject of the invention is also a cosmetic skincare process, more particularly for facial skin, in particular wrinkled skin, comprising the topical application to the skin of a composition as defined above.

The composition and the process according to the invention are in particular intended for smoothing out human facial and/or bodily skin and/or for reducing or effacing the signs of ageing of the skin, in particular for reducing or effacing skin wrinkles and/or fine lines.

The constituents of the composition according to the invention will now be described in greater detail.

The term “tensioning agent” that may be used according to the invention means compounds capable of having a tensioning effect on the skin, i.e. capable of making the skin taut.

In general and according to this first embodiment, the term “tensioning agent” according to the invention means any compound that is soluble or dispersible in water at a temperature ranging from 25° C. to 50° C. at a concentration of 7% by weight in water or at the maximum concentration at which they form a medium of uniform appearance and producing at this concentration of 7% or at this maximum concentration in water a shrinkage of more than 15% in the test described below.

The maximum concentration at which they form a medium of uniform appearance is determined to within ±10% and preferably to within ±5%.

The expression “medium of uniform appearance” means a medium that does not contain any aggregates that are visible to the naked eye.

For the determination of the said maximum concentration, the tensioning agent is gradually added to the water with deflocculating stirring at a temperature ranging from 25° C. to 50° C., and the mixture is then stirred for one hour. The mixture thus prepared is then examined after 24 hours to see if it is of uniform appearance (absence of aggregates visible to the naked eye).

The tensioning effect may be characterized by an in vitro shrinkage test.

A homogeneous mixture of the tensioning agent in water, at a concentration of 7% by weight or at the maximum concentration defined above, is prepared beforehand and as described previously.

30 μl of the homogeneous mixture are placed on a rectangular specimen (10×40 mm, thus having an initial width L₀ of 10 mm) of elastomer with an elastic modulus of 20 MPa and a thickness of 100 μm.

After drying for 3 hours at 22±3° C. and 40±10% relative humidity RH, the elastomer specimen has a shrunken width, noted L₃ₕ, due to the tension exerted by the applied tensioning agent.

The tensioning effect (TE) of the said agent is then quantified in the following manner:

$${ }^{‵} {TE}^{′} = ( {L}_{0} - {L}_{3 h} / {L}_{0} ) \times 100 as \%$$

$$with {L}_{0} = initial width 10 mm$$

$$and {L}_{3 h} = width after 3 hours of drying$$

The tensioning agent may be chosen from:

a) plant proteins and hydrolysates thereof;
b) colloidal particles of mineral fillers;
c) synthetic polymers;
and mixtures thereof.

A person skilled in the art will know how to select, from the chemical categories listed above, the materials corresponding to the tensioning test as described previously.

These different categories of tensioning agents are now described below.

### a) Plant Proteins and Hydrolysates Thereof

Examples of plant proteins and plant protein hydrolysates that may be used as tensioning agents according to the invention are constituted by proteins and protein hydrolysates of corn, rye, wheat, buckwheat, sesame, spelt, tobacco, pea, bean, lentil, soybean, almond and lupin.

Soybean protein is preferably used, such as the products sold under the names Eleseryl SH/VE G 8 and Eleseryl SH VEG 8B by the company Laboratoires Sérobiologiques (INCI name: Glycine Soja Soybean Protein).

### b) Colloidal Particles of Silica or of Silica-Alumina Composite

The term “colloidal particles” means particles dispersed in an aqueous, aqueous-alcoholic or alcoholic medium, preferably an aqueous medium, and having a number-average diameter of between 0.1 and 100 nm and preferably between 3 and 30 nm.

The colloidal particles according to the invention have no thickening property in water, alcohol, or oil or any other solvent. At a concentration of greater than or equal to 15% by weight in water, the viscosity of the solutions thus obtained is less than 0.05 Pa·s for a shear rate equal to 10 s⁻¹. The measurements are taken at 25° C. using a Haake RheoStress RS150 rheometer in cone-plate configuration, the dimensions of the measuring cone being: diameter: 60 mm, and angle: 2°.

These particles are generally prepared according to a sol-gel process and thus differ especially from fumed silica particles, which agglomerate in water to form larger-sized aggregates.

### Colloidal Silica Particles

For the purposes of the patent application, the term “colloidal silicas” means colloidal silica particles dispersed in an aqueous, aqueous-alcoholic or alcoholic medium. Colloidal silica particles have a diameter ranging from 0.1 to 100 nm and preferably from 3 to 30 nm. These particles are in the form of aqueous dispersions and have no thickening property in water, alcohol, or oil or any other solvent. At a concentration of greater than or equal to 15% by weight in water, the viscosity of the solutions thus obtained is less than 0.05 Pa·s for a shear rate equal to 10 s⁻¹. The measurements are taken at 25° C. using a Haake RheoStress RS150 rheometer in cone-plate configuration, the dimensions of the measuring cone being: diameter: 60 mm, and angle: 2°.

As colloidal silicas that may be used in the composition according to the invention, examples that may be mentioned include those sold by the company Catalysts & Chemicals under the names Cosmo S-40 and Cosmo S-50.

### Silica-Alumina Composite Colloidal Particles

The colloidal particles of mineral fillers that may be used according to the invention may also be chosen from silica-alumina composite colloidal particles. The term “silica-alumina composite” means silica particles in which aluminium atoms have been partly replaced with silicon atoms. For the purposes of the patent application, the term “colloidal particles” means colloidal particles dispersed in an aqueous, aqueous-alcoholic or alcoholic medium. Silica-alumina composite colloidal particles have a diameter ranging from 0.1 to 100 nm and preferably from 3 to 30 nm. These particles are in the form of aqueous dispersions and have no thickening property in water, alcohol, oil or any other solvent. At a concentration of greater than or equal to 15% by weight in water, the viscosity of the solutions thus obtained is less than 0.05 Pa·s for a shear rate equal to 10 s⁻¹. The measurements are taken at 25° C. using a Haake RheoStress RS150 rheometer in cone-plate configuration, the dimensions of the measuring cone being: diameter: 60 mm, and angle: 2°.

At a pH of 7, the silica-alumina composite colloidal particles according to the invention have a zeta potential of less than −20 mV and preferably less than −25 mV. The measurements are taken at 25° C. using a Delsa 440SX machine from Coulter Scientific Instrument.

As silica-alumina composite colloidal particles that may be used in the compositions according to the invention, examples that may be mentioned include those sold by the company Grace under the names Ludox AM, Ludox AM-X 6021, Ludox HSA and Ludox TMA.

### e) Synthetic Polymers

The synthetic polymers used according to the invention may be in solution or in suspension in a polar or apolar liquid (latex), especially in aqueous solution or aqueous dispersion, or in dry form redispersible in a cosmetic solvent.
grafted silicone polymers;

The synthetic polymers according to the invention may be chosen especially from interpenetrating polymer networks (IPNs).

These polymers may especially be in the form of statistical linear copolymers, interpenetrating polymer networks (IPNs), polycondensates, grafted silicone polymers and block polymers. Irrespective of its nature, the synthetic polymeric tensioning agent may have a weight-average mass Mw ranging from 3000 to 1 000 000 Da.

### Statistical Linear Copolymers

The tensioning statistical linear copolymers according to the present invention are chosen from statistical copolymers with a linear main chain of ethylenic nature with a molecular weight of less than 600 000 Da (g/mol) and preferably with a weight-average molecular weight of between 15 000 and 600 000 g/mol and containing at least 70% of a monomer with a glass transition temperature Tg of greater than 40° C. (preferably >60° C.), the corresponding homopolymer of which is insoluble in water at 25° C. and at least one ionic hydrophilic monomer. This copolymer may also contain a minor monomer with a Tg of less than 40° C.

These copolymers generally have an overall glass transition temperature of greater than or equal to 45° C.

The copolymers preferably used are those constituted of:
70% to 90% by weight of at least one monomer chosen from styrene, benzyl acrylate, cyclohexyl acrylate, tert-butyl acrylate, isobornyl acrylate, norbornyl acrylate, methyl methacrylate, ethyl methacrylate, isobutyl methacrylate, cyclohexyl methacrylate, benzyl methacrylate, tert-butyl methacrylate, isobornyl methacrylate and norbornyl methacrylate;
10% to 30% by weight of (meth)acrylic acid.

Preferentially, the copolymers used are constituted of:
70% to 90% by weight of at least one monomer chosen from styrene, methyl methacrylate and cyclohexyl methacrylate;
10% to 30% by weight of (meth)acrylic acid.

Among the polymers mentioned above, the ones that will be particularly preferred are:
methyl methacrylate/methacrylic acid copolymers; methyl methacrylate/acrylic acid copolymers, the said copolymers containing between 70% and 90% by weight of methyl methacrylate;
ethyl methacrylate/methacrylic acid copolymers; ethyl methacrylate/acrylic acid copolymers, the said copolymers containing between 70% and 90% by weight of ethyl methacrylate;
isobutyl methacrylate/methacrylic acid copolymers; isobutyl methacrylate/acrylic acid copolymers, the said copolymers containing between 70% and 90% by weight of isobutyl methacrylate; benzyl methacrylate/methacrylic acid copolymers; benzyl methacrylate/acrylic acid copolymers, the said copolymers containing between 70% and 90% by weight of benzyl methacrylate;
benzyl acrylate/methacrylic acid copolymers; benzyl acrylate/acrylic acid copolymers, the said copolymers containing between 70% and 90% by weight of benzyl acrylate;
cyclohexyl methacrylate/methacrylic acid copolymers; cyclohexyl methacrylate/acrylic acid copolymers, the said copolymers containing between 70% and 90% by weight of cyclohexyl methacrylate;
cyclohexyl acrylate/methacrylic acid copolymers; cyclohexyl acrylate/acrylic acid copolymers, the said copolymers containing between 70% and 90% by weight of cyclohexyl acrylate;
tert-butyl methacrylate/methacrylic acid copolymers; tert-butyl methacrylate/acrylic acid copolymers, the said copolymers containing between 70% and 90% by weight of tert-butyl methacrylate;
tert-butyl acrylate/methacrylic acid copolymers; tert-butyl acrylate/acrylic acid copolymers, the said copolymers containing between 70% and 90% by weight of tert-butyl acrylate;
isobornyl methacrylate/methacrylic acid copolymers; isobornyl methacrylate/acrylic acid copolymers, the said copolymers containing between 70% and 90% by weight of isobornyl methacrylate;
isobornyl acrylate/methacrylic acid copolymers; isobornyl acrylate/acrylic acid copolymers, the said copolymers containing between 70% and 90% by weight of isobornyl acrylate;
norbornyl methacrylate/methacrylic acid copolymers; norbornyl methacrylate/acrylic acid copolymers, the said copolymers containing between 70% and 90% by weight of norbornyl methacrylate;
norbornyl acrylate/methacrylic acid copolymers; norbornyl acrylate/acrylic acid copolymers, the said copolymers containing between 70% and 90% by weight of norbornyl acrylate; and
styrene/methacrylic acid copolymers; styrene/acrylic acid copolymers, the said copolymers containing between 70% and 90% by weight of styrene.

The acrylic polymers sold under the names Neocryl XK 90 and Neocryl XK 99 by the company Avecia Neoresins may also be used.

### Interpenetrating Polymers

For the purposes of the present invention, the term “interpenetrating polymer network” means a mixture of two interlaced polymers, obtained by simultaneous polymerization and/or crosslinking of two types of monomer, the mixture obtained having a specific glass transition temperature.

For further details regarding IPNs, reference may be made to the publication by Frisch et al., Polym. Sci. Technol., 4: 395-414 (1974).

These interpenetrating polymer networks, or IPNs, are generally obtained by synthesis and/or crosslinking of the two polymers constituting them in the presence of each other, starting with monomers or prepolymers. In the case of a simultaneous polymerization of two types of monomer, the multifunctional monomers are chosen such that the polymerization mechanisms are different, so that the chain reactions develop independently of each other. When the two polymers are crosslinked, the product is a true IPN; crosslinking of only one of the polymers leads to a semi-IPN. In addition, the two polymers may be crosslinked with each other. These are then referred to as covalent IPNs.

Another process for preparing IPNs, known as the sequential process, consists in polymerizing a first monomer mixture in the presence of a crosslinking agent and of a preformed crosslinked polymer that is soaked with the monomer mixture and thus gradually swells as the polymerization reaction proceeds. The preformed crosslinked polymer must have a degree of crosslinking such that it can absorb at least ten times its weight of liquid.

IPNs are constituted of two intimately interlaced polymers, forming a three-dimensional network that cannot be associated by physical manipulation without breaking covalent bonds. The morphology of the network depends on the competition between the network formation kinetics and the phase separation kinetics.

Examples of IPNs that are suitable for use in the present invention, and also the process for preparing them, are described in patents U.S. Pat. No. 4,644,030 and U.S. Pat. No. 5,173,526, for example.

Preferably, the IPN according to the invention comprises at least one acrylic polymer and more preferentially also comprises at least one polyurethane.

According to one preferred form, the IPN according to the invention comprises a polyurethane polymer and a polyacrylic polymer. Such IPNs are especially those of the Hybridur® series that are commercially available from the company Air Products.

An IPN that is particularly preferred is in the form of an aqueous dispersion of particles with a weight-average size of between 90 and 110 nm and a number-average size of about 80 nm.

The IPN preferably has a glass transition temperature (Tg) range from about −50° C. to +130° C. and preferably from −45° C. to +130° C.

An IPN of a polyurethane copolymer of neopentyl glycol/adipic acid/4,4′-methylenebis(cyclohexyl isocyanate)/dimethylolpropanoic acid and of polymethyl methacrylate is preferably used.

An IPN of this type is especially sold by the company Air Products under the trade name Hybridur® 875 Polymer Dispersion (INCI name: Polyurethane-2 (and) Polymethyl methacrylate), or under the trade names Hybridur® 870 and Hybridur® 880.

### Polycondensate

According to a second variant, the composition may comprise as synthetic polymeric tensioning agent at least one polycondensate. Polymers in the form of polycondensates with a tensioning effect have been described especially in patent application WO 98/29092.

Polycondensates that may be mentioned include polyurethanes, especially anionic, cationic, nonionic or amphoteric polyurethanes, polyurethane-acrylics, polyurethane-polyvinylpyrrolidones, polyester-poly-urethanes, polyether-polyurethanes and polyureas, and mixtures thereof.

The polyurethane may be, for example, an aliphatic, cycloaliphatic or aromatic polyurethane, or polyurea/urethane or polyurea copolymer, comprising, alone or as a mixture:
at least one block of linear or branched aliphatic and/or cycloaliphatic and/or aromatic polyester origin, and/or
at least one block of aliphatic and/or cyclo-aliphatic and/or aromatic polyether origin, and/or
at least one block comprising fluoro groups.

The polyurethanes may also be obtained from branched or unbranched polyesters, or from alkyds comprising labile hydrogens that are modified by reaction with a diisocyanate and a difunctional (for example dihydro, diamino or hydroxyamino) organic compound, also comprising either a carboxylic acid or carboxylate group, or a sulfonic acid or sulfonate group, or alternatively a neutralizable tertiary amine group or a quaternary ammonium group. Mention may also be made of polyesters, polyesteramides, fatty-chain polyesters, polyamides and epoxyester resins.

For the purpose of forming a polyurethane, as monomers bearing an anionic group that may be used in the polycondensation, mention may be made of dimethylol-propionic acid, trimellitic acid or a derivative such as trimellitic anhydride, the sodium salt of pentane-diol-3-sulfonic acid or the sodium salt of 5-sulfo-1,3-benzenedicarboxylic acid.

Among the polycondensates that may be mentioned are the polymers sold under the trade names Avalure UR410, Avalure UR405 and Avalure UR460 by the company Noveon, and under the trade names Neorez R974, Neorez R981 and Neorez R970 by the company Avecia.

Mention may also be made of combinations of polymers, such as polyurethanes with a degree of shrinkage or equal to 20% and acrylic polymers with a degree of shrinkage or equal to 20%, described in patent application WO 2005/067 884.

### Grafted Silicone Polymer

Among the synthetic polymeric tensioning agents used in the composition according to the invention, mention may be made, as a variant, of grafted silicone polymers, especially as defined in patent application EP-1 038 519. Such a polymer may be more particularly a polymer comprising a silicone or polysiloxane (Si—O-polymer) main chain onto which is grafted, within the said chain and also optionally on at least one of its ends, at least one organic group not comprising silicone.

The polymers containing a polysiloxane backbone grafted with non-silicone organic monomers, according to the invention, can be existing commercial products or alternatively can be obtained according to any means known to those skilled in the art, in particular by reaction between (i) a starting silicone which is correctly functionalized on one or more of its silicon atoms, and (ii) a non-silicone organic compound which is itself correctly functionalized with a function which is capable of reacting with the functional group(s) borne by the said silicone, forming a covalent bond; a classic example of such a reaction is the hydrosilylation reaction between ≡Si—H groups and vinyl groups CH₂═CH—, or alternatively the reaction between thio functional groups —SH with these same vinyl groups.

Examples of polymers containing a polysiloxane backbone grafted with non-silicone organic monomers that are suitable for carrying out the present invention, and also their particular mode of preparation, are described in particular in patent applications EP-A-0 582 152, WO 93/23009 and WO 95/03776, the teachings of which are included in their entirety in the present description by way of non-limiting references.

According to a particularly preferred embodiment of the present invention, the silicone polymer containing a polysiloxane backbone grafted with non-silicone organic monomers which is used comprises the result of the free-radical copolymerization between, on the one hand, at least one non-silicone ethylenically unsaturated anionic organic monomer and/or a non-silicone ethylenically unsaturated hydrophobic organic monomer, and, on the other hand, a silicone containing in its chain at least one functional group capable of reacting with the said ethylenic unsaturations of the said non-silicone monomers, forming a covalent bond, in particular thio functional groups.

According to the present invention, the said ethylenically unsaturated anionic monomers are preferably chosen, alone or as mixtures, from linear or branched, unsaturated carboxylic acids, optionally partially or totally neutralized in the form of a salt, it being possible for this or these unsaturated carboxylic acid(s) to be, more particularly, acrylic acid, methacrylic acid, maleic acid, maleic anhydride, itaconic acid, fumaric acid and crotonic acid. The suitable salts are, in particular, alkali metal salts, alkaline-earth metal salts and ammonium salts. It will likewise be noted that, in the final grafted silicone polymer, the organic group of anionic nature which comprises the result of the free-radical (homo)polymerization of at least one anionic monomer of unsaturated carboxylic acid type can, after reaction, be post-neutralized with a base (sodium hydroxide, aqueous ammonia, etc.) in order to place it in the form of a salt.

According to the present invention, the ethylenically unsaturated hydrophobic monomers are preferably chosen, alone or as mixtures, from acrylic acid esters of alkanols and/or methacrylic acid esters of alkanols. The alkanols are preferably C₁-C₁₈ and more particularly C₁-C₁₂. The preferred monomers are chosen from the group consisting of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate and stearyl (meth)acrylate, or mixtures thereof.

One family of silicone polymers containing a polysiloxane backbone grafted with non-silicone organic monomers that is particularly suitable for carrying out the present invention consists of silicone polymers comprising in their structure the unit of formula (I) below:

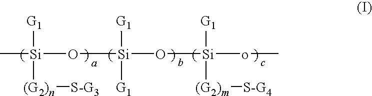

in which the radicals G₁, which may be identical or different, represent hydrogen, a C₁-C₁₀ alkyl radical or a phenyl radical; the radicals G₂, which may be identical or different, represent a C₁-C₁₀ alkylene group; G₃ represents a polymer residue resulting from the (homo)polymerization of at least one ethylenically unsaturated anionic monomer; G₄ represents a polymer residue resulting from the (homo)polymerization of at least one ethylenically unsaturated hydrophobic monomer; m and n are, independently of each other, equal to 0 or 1; a is an integer ranging from 0 to 50; b is an integer which may be between 10 and 350, c is an integer ranging from 0 to 50; with the proviso that one of the parameters a and c is other than 0.

Preferably, the unit of formula (I) above has at least one, and even more preferentially all, of the following characteristics:
the radicals G₁ denote a C₁-C₁₀ alkyl radical;
n is not zero, and the radicals G₂ represent a divalent C₁-C₃ radical;
G₃ represents a polymer radical resulting from the (homo)polymerization of at least one monomer of the ethylenically unsaturated carboxylic acid type, preferably acrylic acid and/or methacrylic acid;
G₄ represents a polymer radical resulting from the (homo)polymerization of at least one monomer of the C₁-C₁₀ alkyl (meth)acrylate type.

Examples of grafted silicone polymers corresponding to formula (I) are thus, especially, polydimethylsiloxanes (PDMSs) onto which are grafted, via a connecting chain of thiopropylene type, polymer units of the poly(meth)acrylic acid type and/or of the polyalkyl (meth)acrylate type, especially of a C₁-C₃ or even C₁ alkyl.

These polymers are referenced under the CTFA name Polysilicone-8.

It may thus be a case of a polydimethylsiloxane grafted with propylthio(polymethyl acrylate/methyl methacrylate/methacrylic acid) or a polydimethyl-siloxane grafted with propylthio(polymethyl acrylate), propylthio(polymethyl methacrylate) and propylthio(polymethacrylic acid). As a variant, it may be a polydimethylsiloxane grafted with propylthio(polyisobutyl methacrylate) and propylthio(polymethacrylic acid). A polydimethyl-siloxane grafted with propylthio(polymethyl acrylate/methyl methacrylate/methacrylic acid) is preferably used.

A polymer of this type is especially available under the trade name VS 80, VS 70 (at 10% in water) or LO 21 (in pulverulent form) from the company 3M.

Preferably, the number-average molecular mass of the silicone polymers containing a polysiloxane backbone grafted with non-silicone organic monomers of the invention ranges from 10 000 to 1 000 000 approximately and even more preferentially from 10 000 to 100 000 approximately.

### Block Polymer

As a variant, the synthetic polymeric tensioning agents that may be used in the composition according to the invention may be polystyrene (PS)-polyethyl acrylate (PEA) block polymers.

Very generally speaking, the term “block polymer” means a polymer constituted of at least two distinct homopolymers constituted solely of monomers A and B, respectively. Thus, the blocks according to the invention are, respectively, polystyrene (PS) and polyethyl acrylate (PEA) blocks.

In the context of this variant, the polymer may be a triblock polymer of PS-PEA-PS type or a multiblock polymer of PS-[PEA-PS]n or PEA-[PS-PEA]n type, in which n is a positive integer and is preferably equal to 1. Advantageously, these block polymers are linear copolymers. The molecular weight of this polymer is preferably greater than 10 000 daltons and even more preferably greater than 50 000 daltons. The weight ratio of the PS and PEA monomers may be defined such that PS/PEA is greater than 1 and preferably such that PS/PEA is greater than 5.

Mention may be made of the triblock polymer PS(30 000)-PEA(10 000)-PS(30 000), which is most particularly suitable for use in the invention. This particularly advantageous block copolymer is a triblock copolymer comprising:
a first block comprising units derived from styrene, with a number-average molecular mass of 30 000 g/mol;
a second block constituted of units derived from ethyl acrylate, with a number-average molecular mass of 10 000 g/mol;
a third block comprising units derived from styrene, with a number-average molecular mass of 30 000 g/mol.

A copolymer corresponding to the definition given above may be a copolymer for which the first block and/or the third block, and preferably the first block and the third block, comprises, besides units derived from styrene, units derived from methacrylic acid, for example in a (styrene/methacrylic acid) mass ratio of 98/2.

The synthetic copolymers used according to the invention may also as a variant be constituted of a polystyrene-polyethyl acrylate statistical copolymer. The weight ratio of the PS and PEA monomers is defined therein such that PS/PEA>1 and preferably such that PS/PEA>5.

Alternatively, the tensioning polymers according to the invention may also be chosen from vinyl derivatives such as polyvinyl alcohols and polyvinylpyrrolidones, whether they are in block or statistical form.

According to one preferred mode of the invention, a tensioning agent will be used that is chosen from:
interpenetrating polymer networks comprising a polyurethane and an acrylic polymer, and in particular the product sold under the name Hybridur 875 by the company Air Products;
colloidal particles of silica or of silica-alumina composite;
polydimethylsiloxanes (PDMS) onto which are grafted, via a connecting unit of thiopropylene type, polymer units of the poly(meth)acrylic acid type and/or of the polyalkyl (meth)acrylate type, especially of a C₁-C₃ or even C₁ alkyl.

Advantageously, the preferred tensioning agent is chosen from interpenetrating polymers, in particular comprising a polyurethane polymer and an acrylic polymer, such as the products sold under the name Hybridur and in particular Hybridur 875.

Another preferred tensioning agent may be chosen from colloidal particles of silica or of silica-alumina composite.

According to another embodiment according to the invention, a propylthio(polyethyl acrylate/methyl methacrylate/methacrylic acid)-grafted polydimethyl-siloxane may be used as tensioning agent.

The tensioning agent may be present in the composition according to the invention in a content ranging from 0.1% to 10% by weight, preferably ranging from 1% to 10% by weight and preferentially ranging from 1% to 8% by weight relative to the total weight of the composition.

The composition according to the invention also comprises an acrylic polymer containing a group of phosphorylcholine type, which will be referred to in the rest of the description as an acrylic PC polymer.

Documents U.S. Pat. No. 5,468,475, EP-A-767 212 and EP-A-1 163 905 disclose aqueous compositions containing an acrylic polymer containing a group of phosphorylcholine type, especially moisturizing cosmetic skincare compositions. However, these documents do not describe combining it with a skin-tensioning agent.

The acrylic PC polymer is advantageously a water-soluble or water-dispersible polymer, and preferably a water-soluble polymer.

The term “water-soluble polymer” means a polymer with a solubility of at least 0.1% by weight in water at 25° C.

The term “water-dispersible polymer” means a polymer that is capable of dispersing uniformly in water at 25° C., without forming a non-uniform phase.

The term “acrylic polymer containing a group of phosphorylcholine type” means a polymer having an acrylic backbone and comprising pendent groups (or side chains) containing a group of formula (I) below:

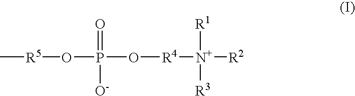

in which R¹, R² and R³ independently denote an alkyl group containing from 1 to 8 carbon atoms; R⁴ denotes —(CH₂—CHR₆O)ₘ—(CH₂—CHR₆)ₚ— with R₆ denoting a hydrogen atom or a methyl or ethyl group, m denoting an integer ranging from 0 to 10, and p denoting an integer ranging from 1 to 2; R⁵ denotes —(CH₂)_{g}—, g being an integer ranging from 2 to 10.

Such a polymer may be obtained by polymerization of an acrylic monomer comprising the group of formula (I) described previously, which will be referred to in the rest of the description as the acrylic PC monomer.

Advantageously, the acrylic PC monomer is a monomer corresponding to formula (II) below:

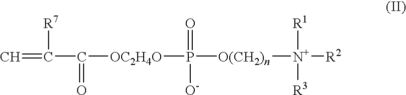

in which R¹, R² and R³ independently denote an alkyl group containing from 1 to 8 carbon atoms; n represents an integer ranging from 2 to 4; R⁷ denotes a hydrogen atom or a methyl group.

Acrylic PC monomers that may be mentioned include the following monomers:
2-(meth)acryloyloxyethyl-2′-(trimethylammonio)ethyl phosphate,
3-(meth)acryloyloxypropyl-2′-(trimethylammonio)ethyl phosphate,
4-(meth)acryloyloxybutyl-2′-(trimethylammonio)ethyl phosphate,
5-(meth)acryloyloxypentyl-2′-(trimethylammonio)ethyl phosphate,
2-(meth)acryloyloxyethyl-2′-(triethylammonio)ethyl phosphate,
3-(meth)acryloyloxypropyl-2′-(triethylammonio)ethyl phosphate,
4-(meth)acryloyloxybutyl-2′-(triethylammonio)ethyl phosphate,
5-(meth)acryloyloxypentyl-2′-(triethylammonio)ethyl phosphate,
2-(meth)acryloyloxyethyl-2′-(tripropylammonio)ethyl phosphate,
3-(meth)acryloyloxypropyl-2′-(tripropylammonio)ethyl phosphate,
4-(meth)acryloyloxybutyl-2′-(tripropylammonio)ethyl phosphate,
5-(meth)acryloyloxypentyl-2′-(tripropylammonio)ethyl phosphate,
2-(meth)acryloyloxyethyl-2′-(tributylammonio)ethyl phosphate,
3-(meth)acryloyloxypropyl-2′-(tributylammonio)ethyl phosphate,
4-(meth)acryloyloxybutyl-2′-(tripropylammonio)ethyl phosphate,
5-(meth)acryloyloxypentyl-2′-(tributylammonio)ethyl phosphate,
2-(meth)acryloyloxyethyl-3′-(trimethylammonio)propyl phosphate,
2-(meth)acryloyloxyethyl-3′-(triethylammonio)propyl phosphate,
2-(meth)acryloyloxyethyl-4′-(triethylammonio)butyl phosphate,
2-(meth)acryloyloxyethyl-3′-(tripropylammonio)propyl phosphate,
2-(meth)acryloyloxyethyl-4′-(tripropylammonio)butyl phosphate,
2-(meth)acryloyloxyethyl-3′-(tributylammonio)propyl phosphate,
2-(meth)acryloyloxyethyl-4′-(tributylammonio)butyl phosphate,
3-(meth)acryloyloxyethyl-3′-(trimethylammonio)propyl phosphate,
3-(meth)acryloyloxypropyl-4′-(trimethylammonio)butyl phosphate,
3-(meth)acryloyloxypropyl-3′-(triethylammonio)propyl phosphate,
3-(meth)acryloyloxypropyl-4′-(triethylammonio)butyl phosphate,
3-(meth)acryloyloxypropyl-3′-(tripropylammonio)propyl phosphate,
3-(meth)acryloyloxypropyl-4′-(tripropylammonio)butyl phosphate,
3-(meth)acryloyloxypropyl-3′-(tributylammonio)propyl phosphate,
3-(meth)acryloyloxypropyl-4′-(tributylammonio)butyl phosphate,
4-(meth)acryloyloxybutyl-3′-(trimethylammonio)propyl phosphate,
4-(meth)acryloyloxybutyl-4′-(trimethylammonio)butyl phosphate,
4-(meth)acryloyloxybutyl-4′-(triethylammonio)butyl phosphate,
4-(meth)acryloyloxybutyl-3′-(tripropylammonio)propyl phosphate,
4-(meth)acryloyloxybutyl-4′-(tripropylammonio)butyl phosphate,
4-(meth)acryloyloxybutyl-3′-(tributylammonio)propyl phosphate, and
4-(meth)acryloyloxybutyl-4′-(tributylammonio)butyl phosphate.

2-(Meth)acryloyloxyethyl-2′-(triethylammonio)ethyl phosphate, also known as 2-(methacryloyloxyethyl)-phosphorylcholine, is preferably used as acrylic PC monomer.

Preferably, the acrylic PC monomer used according to the invention is a polymer obtained by polymerization of an acrylic PC monomer as described previously and optionally of one or more additional monomers other than the acrylic PC monomer.

The additional monomers may be chosen from methyl (meth)acrylate, ethyl (meth)acrylate, butyl (meth)acrylate, (meth)acrylic acid, (meth)acrylamide, 2-hydroxyethyl (meth)acrylate, ethyl vinyl ether, butyl vinyl ether, N-vinylpyrrolidone, vinyl chloride, ethylene, isobutylene, acrylonitrile, styrene, methylstyrene and chloromethylstyrene.

The acrylic PC polymer may comprise from 40 mol % to 100 mol % of units derived from the acrylic PC monomer as described previously and from 0 to 60 mol % of units derived from the additional monomer.

Preferably, the polymer containing a group of phosphorylcholine type is chosen from the 2-(methacryloyloxyethyl)phosphorylcholine homopolymer, the 2-(methacryloyloxyethyl)phosphorylcholine/butyl methacrylate copolymer, the 2-(methacryloyloxyethyl)-phosphorylcholine/2-hydroxy-3-methacryloyloxypropyl-trimethylammonium chloride copolymer, the 2-(methacryl-oyloxyethyl)phosphorylcholine/butyl methacrylate/sodium methacrylate terpolymer and the 2-(methacryloyloxy-ethyl)phosphorylcholine/stearyl methacrylate copolymer.

The 2-(methacryloyloxyethyl)phosphorylcholine homo-polymer or the 2-(methacryloyloxyethyl)phosphoryl-choline/butyl methacrylate copolymer is preferably used, and more preferentially the 2-(methacryloyloxy-ethyl)phosphorylcholine homopolymer.

Such polymers are described in documents EP-A-1 163 905, EP-A-1 095 665, FR-A-2 698 003 and EP-A-767 212, the content of which is incorporated by way of reference into the present patent application.

The acrylic PC polymer preferably has a weight-average molecular weight ranging from 50 000 to 1 000 000 and preferentially ranging from 80 000 to 800 000.

The following may be used as acrylic polymer containing a phosphorylcholine group in accordance with the invention:
poly-2-(methacryloyloxyethyl)phosphorylcholine at 40% in a water/butanediol mixture (5% butanediol), sold under the name Lipidure HM by the company Nippon Oil and Fats; this product has the CTFA name: polyphosphorylcholine glycol acrylate (and) butylene glycol;
the 2-(methacryloyloxyethyl)phosphorylcholine/butyl methacrylate copolymer (90/10) as a 5% solution in water, sold under the name Lipidure PMB by the company Nippon Oil and Fats; this product has the CTFA name: Polyquaternium-51;
the 2-(methacryloyloxyethyl)phosphorylcholine/2-hydroxy-3-methacryloyloxypropyltrimethylammonium chloride copolymer as a 5% solution in water, sold under the name Lipidure-C by the company Nippon Oil and Fats;
the 2-(methacryloyloxyethyl)phosphorylcholine/butyl methacrylate/sodium methacrylate terpolymer as a 5% solution in water, sold under the name Lipidure-A by the company Nippon Oil and Fats;
the 2-(methacryloyloxyethyl)phosphorylcholine/stearyl methacrylate copolymers sold under the names Lipidure-S, Lipidure-NR and Lipidure-NA by the company Nippon Oil and Fats; these products have the CTFA name: Polyquaternium-61.

The acrylic PC polymer may be present in the composition according to the invention in a content ranging from 0.01% to 20% by weight, preferably ranging from 0.1% to 10% by weight and preferentially ranging from 0.1% to 5% by weight relative to the total weight of the composition.

The composition according to the invention is generally suited to topical application to the skin and thus generally comprises a physiologically acceptable medium, i.e. a medium that is compatible with the skin and/or its integuments. It is preferably a cosmetically acceptable medium, i.e. a medium that has a pleasant colour, odour and feel and that does not cause any unacceptable discomfort (stinging, tautness or redness) liable to put the consumer off using this composition.

The composition according to the invention may be in any galenical form conventionally used for topical application, and especially in the form of dispersions of the lotion or aqueous gel type, emulsions of liquid or semi-liquid consistency of the milk type, obtained by dispersing a fatty phase in an aqueous phase (O/W) or, conversely, (W/O), or suspensions or emulsions of soft, semi-solid or solid consistency of the cream or gel type, or alternatively multiple emulsions (W/O/W or O/W/O), microemulsions, vesicular dispersions of ionic and/or nonionic type, or wax/aqueous phase dispersions. These compositions are prepared according to the usual methods.

According to one preferred embodiment of the invention, the composition is in the form of an O/W emulsion or an aqueous gel.

This composition may also contain various adjuvants commonly used in cosmetics, such as emulsifiers, for instance fatty acid esters of polyethylene glycol, optionally polyoxyethylenated fatty acid esters of sorbitan, polyoxyethylenated fatty alcohols, and fatty acid esters or ethers of sugars such as sucrose or glucose; fillers; preserving agents; sequestrants; fragrances; and thickeners and/or gelling agents, in particular polyacrylamides, acrylic homopolymers and copolymers, and acrylamidomethylpropanesulfonic acid homopolymers and copolymers.

Needless to say, a person skilled in the art will take care to select this or these optional additional compound(s) and/or the amount thereof such that the anti-wrinkle properties of the composition according to the invention are not, or are not substantially, adversely affected by the envisaged addition.

The composition according to the invention may also contain anti-ageing active agents with an effect complementary to the combination according to the invention, such as at least one compound chosen from desquamating agents, moisturizers, agents for stimulating keratinocyte proliferation and/or differentiation, agents for stimulating collagen and/or elastin synthesis or for preventing their degradation, depigmenting agents, anti-glycation agents, agents for stimulating glycosaminoglycan synthesis, dermo-decontracting agents or muscle relaxants, antioxidants and free-radical scavengers, and mixtures thereof.

Examples of such active agents are: retinol and derivatives thereof such as retinyl palmitate; ascorbic acid and derivatives thereof such as magnesium ascorbyl phosphate and ascorbyl glucoside; tocopherol and derivatives thereof such as tocopheryl acetate; nicotinic acid and precursors thereof such as nicotinamide; ubiquinone; glutathione and precursors thereof such as L-2-oxothiazolidine-4-carboxylic acid; plant extracts and especially extracts of sea fennel and of olive leaf; algal extracts and in particular of laminaria; bacterial extracts; sapogenins such as diosgenin and extracts of Dioscorea plants, in particular of wild yam, containing them; α-hydroxy acids; β-hydroxy acids, such as salicylic acid and 5-n-octanoylsalicylic acid; oligopeptides and pseudodi-peptides and acyl derivatives thereof, in particular {2-[acetyl (3-trifluoromethylphenyl)amino]-3-methyl-butyrylamino}acetic acid and the lipopeptides sold by the company Sederma under the trade names Matrixyl 500 and Matrixyl 3000; lycopene; and mixtures thereof.

The composition according to the invention is applied according to the usual techniques, for example by application (especially of creams, gels, sera or lotions) to the skin intended to be treated, in particular the skin of the face and/or the neck, especially the skin around the eyes. In the context of this process, the composition may be, for example, a care composition or a makeup composition, in particular a foundation.

The invention will now be described with reference to the following examples, which are given as non-limiting illustrations.

### EXAMPLES

### Example 1

### Demonstration of Maintenance of the Tensioning Effect in the Presence of an Acrylic Polymer Containing a Group of Phosphorylcholine Type

The behaviour of two tensioning agents (Ludox Amx6021 colloidal silica from Grace Davison and Hybridur 875 interpenetrating polymer network from Air Products) was studied. These tensioning agents were formulated as an aqueous solution with a content of 7% by weight of active material alone or in the presence of 3% glycerol or 0.2% acrylic polymer containing a phosphorylcholine group (Lipidure HM from Nippon Oil and Fats).

The tensioning power of each composition was measured in vitro by placing 30 μl of the evaluated solution onto a rectangular (10×40 mm) elastomeric specimen having an elastic modulus of about 20 MPa and a thickness of 100 μm. After drying for 3 hours at 22±3° C. and 40±10% relative humidity, the width of the specimen at its centre (L₃ₕ) is measured.

The tensioning effect (TE) is quantified in the following manner:

$${ }^{‵} {TE}^{′} = ( {L}_{0} - {L}_{3 h} / {L}_{0} ) \times 100 as \%$$

$$with {L}_{0} = initial width 10 mm$$

$$and {L}_{3 h} = width after 3 hours of drying$$

The following results were obtained:

| | TE = (L₀ − L_{3 h}/L₀) × 100 in % | |
|---|---|---|
| | Ludox AMX6021 (1) | Hybridur 875 (2) |
| Tensioning agent | 47.6% | 66.3% |
| (7% AM in water) | | |
| Tensioning agent (7% AM) + | 3.8% | 6.9% |
| glycerol (3% AM) | | |
| Tensioning agent (7% AM) + | 77.5% | 83.8% |
| Lipidure HM (3) (0.2% AM) | | |

(1) Ludox AMX6021 from Grace Davison: 30% aqueous dispersion of colloidal amorphous silica
(2) Hybridur 875 from Air Products: 40% anionic aqueous dispersion of an interpenetrating network of polyurethane and acrylic polymer
(3) Lipidure HM from Nippon Oil and Fats:
poly-2-(methacryloyloxyethyl)phosphorylcholine at 40% in a
water/butanediol mixture (5% butanediol). (Comment: the
active material content used has a moisturizing
activity equivalent to 3% glycerol).

The results obtained show that the polymer Lipidure HM makes it possible to conserve, or even to improve, the tensioning effect of the two tensioning agents tested, whereas glycerol makes the tensioning effect disappear.

### Example 2

### Anti-Wrinkle Oil-in-Water Emulsion

### Phase A

| | |
|---|---|
| Mixture of glyceryl stearate and of PEG-100 stearate | 2.00 g |
| (Arlacel ® 165 FL from Uniqema) | |
| Mixture of (linear C₁₄-C₁₅) dialkyl tartrate, of cetyl- | 1.50 g |
| stearyl alcohol and of oxyethylenated (25 EO) oxypropyl- | |
| enated (25 PO) lauryl alcohol (Cosmacol PSE from Sasol) | |
| Cyclohexasiloxane | 5.00 g |
| Stearyl alcohol | 1.00 g |

### Phase B

| Water | qs 100 | g |
|---|---|---|
| Preserving agent | qs | |
| Pentasodium salt of ethylenediaminetetramethylene- | 0.05 | g |
| phosphonic acid | | |
| Polyacrylamidomethylpropanesulfonic acid partially | 0.40 | g |
| neutralized with aqueous ammonia and highly | | |
| crosslinked (Hostacerin AMPS from Clariant) | | |
| Xanthan gum | 0.20 | g |

### Phase C

| | |
|---|---|
| Aqueous dispersion of colloidal silica | 17.10 g |
| containing 40% AM (Cosmo S40) | |

### Phase D

| | |
|---|---|
| Aqueous solution of poly-2-(methacryl- | 0.50 g |
| oyloxyethyl)phosphorylcholine at 40% in a | |
| water/butanediol mixture (5% butanediol), | |
| sold under the name Lipidure HM by the | |
| company Nippon Oil and Fats | |

### Procedure:

heat phase B to about 75° C. and incorporate the Hostacerin AMPS therein; stir until a homogeneous gel is obtained;
heat phase A to about 75° C.;
prepare the emulsion by incorporating phase A into phase B;
at 40-45° C., incorporate phase C and then phase D and continue stirring until cooling is complete.

The composition applied to wrinkled areas of the face rapidly effaces the wrinkles and the effect is maintained for several hours.

### Example 3

### Anti-Wrinkle Water-in-Oil Emulsion

### Phase A

| | | |
|---|---|---|
| Cetyldimethicone copolyol (Abil EM 90 from | 1.5 | g |
| Goldschmidt) | | |
| Polyglyceryl-4 isostearate (Isolan GI 34 from | 0.5 | g |
| Goldschmidt) | | |
| Isohexadecane | 4 | g |
| Squalane | 1.85 | g |
| Dimethicone | 2.05 | g |
| Apricot kernel oil | 1.1 | g |
| Cyclopentasiloxane | 9 | g |

| | |
|---|---|
| Preserving agent | qs |

### Phase B

| Water | qs 100 | g |
|---|---|---|
| Propylene glycol | 3 | g |
| Magnesium sulfate | 1.75 | g |
| Preserving agent | qs | |
| Aqueous solution of poly-2-(methacryloyloxy- | 0.50 | g |
| ethyl)phosphorylcholine at 40% in a | | |
| water/butanediol mixture (5% butanediol), sold | | |
| under the name Lipidure HM by the company Nippon | | |
| Oil and Fats | | |

### Phase C

| | |
|---|---|
| Aqueous dispersion containing 40% by weight | 17.1 g |
| of particles of an interpenetrating network | |
| of polyurethane and polyacrylic polymers | |
| (Hybridur ® 875 from Air Products) | |

### Phase D

| | |
|---|---|
| Polyamide powder (Orgasol 2002 D Nat Cos from | 3 g |
| Arkema) | |

### Procedure:

homogenize, at room temperature with stirring, phase A and phase B separately,
prepare the emulsion by incorporating phase B into phase A,
incorporate phases C and D with stirring.

The composition applied to wrinkled facial skin effaces the wrinkles quickly, the effect lasting for several hours.

### Example 4

### Anti-Wrinkle Serum

The following composition was prepared:

| Water | qs 100 | g |
|---|---|---|
| Polyacrylamidomethylpropanesulfonic acid partially | 1.00 | g |
| neutralized with aqueous ammonia and highly | | |
| crosslinked (Hostacerin AMPS from Clariant) | | |
| Preserving agents | qs | |
| Aqueous solution of poly-2-(methacryloyloxy- | 0.50 | g |
| ethyl)phosphorylcholine at 40% in a | | |
| water/butanediol mixture (5% butanediol), sold | | |
| under the name Lipidure HM by the company Nippon | | |
| Oil and Fats | | |
| Aqueous dispersion of colloidal silica containing | 20.00 | g |
| 30% AM (Ludox AMX6021 from Grace Davison) | | |

The serum applied to the face rapidly effaces wrinkles, the effect lasting for several hours.

1. A cosmetic composition comprising:
a physiologically acceptable medium;
an acrylic polymer containing a phosphorylcholine group; and
a skin-tensioning agent;
wherein the skin-tensioning agent is at least one selected from the group consisting of:
plant proteins and hydrolysates thereof;
colloidal particles of silica or of silica-alumina composite; and
at least one synthetic polymer selected from the group consisting of:
i) an interpenetrating polymer network comprising a polyurethane polymer and an acrylic polymer; and
ii) a propylthio (polymethyl acrylate/methyl methacrylate/methacrylic acid)-grafted polydimethyl-siloxane.

2. The composition according to claim 1, wherein the tensioning agent is a soybean protein or a protein hydrolysate.

3. The composition according to claim 1, wherein the tensioning agent is colloidal particles of silica or of silica-alumina composite.

4. The composition according to claim 1, wherein the tensioning agent is a propylthio (polymethyl acrylate/methyl methacrylate/methacrylic acid)-grafted polydimethylsiloxane.

5. The composition according to claim 1, wherein the tensioning agent is a polymer of interpenetrating polymer network type comprising a polyurethane polymer and an acrylic polymer.

6. The composition according claim 5,
wherein the tensioning agent is a polymer of interpenetrating polymer network type comprising a polyurethane copolymer of neopentyl glycol/adipic acid/4,4′-methylenebis (cyclohexyl isocyanate)/dimethylolpropanoic acid and of polymethyl methacrylate.

7. The composition according to claim 1, wherein a content of the tensioning agent from 1% to 10% by weight of active material relative to the total weight of the composition.

8. The composition according to claim 1, wherein the acrylic polymer containing a phosphorylcholine group is a polymer having an acrylic backbone and comprising end groups containing a group of formula (I): 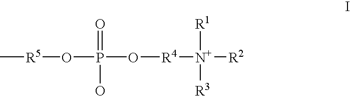
wherein
R¹, R² and R³ independently are an alkyl group containing from 1 to 8 carbon atoms;
R⁴ is —(CH₂—CHR₆O)m-(CH₂—CHR₆)ₚ— wherein R₆ is a hydrogen atom a methyl or ethyl group,
m is an integer ranging from 0 to 10,
p is an integer ranging from 1 to 2;
R⁵ is —(CH₂)_{g}—, and
g is an integer from 2 to 10.

9. The composition according to claim 8, wherein the acrylic polymer containing a phosphorylcholine group is a polymer obtained by polymerization of an acrylic monomer corresponding to formula (II): 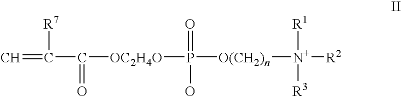
wherein
R¹, R² and R³ independently are an alkyl group containing from 1 to 8 carbon atoms;
n is an integer from 2 to 4;
R⁷ is a hydrogen atom or a methyl group.

10. The composition according to claim 9, wherein the acrylic monomer of formula (II) is 2-(meth)acryloyloxyethyl-2′-(trimethylammonio)-ethyl phosphate.

11. The composition according to claim 8, wherein the acrylic polymer containing phosphorylcholine group is selected from the group consisting of a 2-(methacryloyloxyethyl)phosphorylcholine homopolymer, a 2-(methacryloyloxyethyl)phosphorylcholine/butyl methacrylate copolymer, a 2-(methacryl-oyloxyethyl)phosphorylcholine/2-hydroxy-3-methacryloyl oxypropyltrimethylammonium chloride copolymer, a 2-(methacryloyloxyethyl)phosphorylcholine/butyl methacrylate/sodium methacrylate terpolymer and a 2-(methacryloyloxyethyl)phosphorylcholine/stearyl methacrylate copolymer.

12. The composition according to claim 11, wherein the acrylic polymer containing a phosphorylcholine is the 2-(methacryloyloxyethyl)phosphorylcholine homopolymer.

13. The composition according to claim 1, wherein a content of the acrylic polymer containing a phosphorylcholine group is from 0.01% to 20% by weight, relative to the total weight of the composition.

14. A cosmetic skincare process, comprising:
applying the composition according to claim 1 to the skin under care.

15. The process according to claim 14, wherein the application of the composition to the skin under care is to attenuate wrinkles in the skin.
